(19) United States
(12) Patent Application Publication
(10) Pub. No.: US 2017/0324053 A1
(43) Pub. Date: Nov. 9, 2017

(54) HYBRID ORGANIC-INORGANIC PEROVSKITE-BASED SOLAR CELL WITH COPPER OXIDE AS A HOLE TRANSPORT MATERIAL

(71) Applicant: QATAR FOUNDATION FOR EDUCATION, SCIENCE AND COMMUNITY DEVELOPMENT, DOHA (QA)

(72) Inventors: NOUAR AMOR TABET, DOHA (QA); FAHHAD HUSSAIN ALHARBI, DOHA (QA); MOHAMMAD ISTIAQUE HOSSAIN, DOHA (QA)

(73) Assignee: QATAR FOUNDATION FOR EDUCATION, SCIENCE AND COMMUNITY DEVELOPMENT, DOHA (QA)

(21) Appl. No.: 15/528,093

(22) PCT Filed: Nov. 20, 2015
(86) PCT No.: PCT/QA2015/050002

§ 371 Date: May 18, 2017

Related U.S. Application Data
(60) Provisional application No. 62/082,583, filed on Nov. 20, 2014.

(51) Int. Cl. H01L 51/42; C01G 23/047; C07F 7/24; C07C 211/63; C01G 3/02

(52) U.S. Cl. CPC H01L 51/4226; C01G 23/047; C07F 7/24; C01G 3/02; C07C 211/63

(57) ABSTRACT

The hybrid organic-inorganic perovskite-based solar cell with copper oxide as a hole transport material includes a transparent conducting film layer (12) sandwiched between a glass substrate (11) and a titanium dioxide layer (14). The transparent conducting film layer (12) can be fluorine-doped tin oxide. A lead methylammonium tri-iodide perovskite layer (16) is formed on the titanium dioxide layer (14), such that the titanium dioxide layer (14) is sandwiched between the lead methylammonium tri-iodide perovskite layer (16) and the transparent conducting film layer (12). A layer of copper oxide (Cu2O) (18), as a hole transport material, is formed on the lead methylammonium tri-iodide perovskite layer (16). The lead methylammonium tri-iodide perovskite layer (16) is sandwiched between the layer of hole transport material (18) and the titanium dioxide layer (14). A gold contact (20) is formed on the layer of hole transport material (18).

### TECHNICAL FIELD

The present invention relates to solar cells, and particularly to a hybrid organic-inorganic perovskite-based solar cell with copper oxide as a hole transport material.

### BACKGROUND ART

Methylammonium lead halide perovskites (CH₃NH₃PbX₃, X¼Cl, Br, I) have recently emerged as promising materials for optoelectronics. These materials can be readily processed in solution and are made of abundant elements, thus making them highly desirable in the design of cost effective devices. The use of perovskites as absorbing materials has enabled researchers to design solar cells with power conversion efficiencies exceeding 15%. Additionally, lasers made of perovskites show gains that exceed those of the best devices made of organic materials.

However, there are few challenges that need to be addressed before perovskites replace silicon from its dominant position in the photovoltaic industry. Namely, these include: enhancing the resistance of perovskites to degradation, the replacement of the expensive conventional hole transport material (HTM) made of 2,2′,7,7′-tetrakis(N,N-di-p-methoxyphenylamine)-9′,9-spirobifluorene (spiro-OMeTAD), and the substitution of lead with a non-toxic element. Intensive efforts are being deployed worldwide to tackle the above issues. Moisture and ultraviolet (UV) light have been found to be responsible for the degradation of the lead halide perovskite-based cells. Spiro-OMeTAD HTM has been routinely used in organic photovoltaics (OPV) and organic optoelectronics and, thus, for this reason, it was suggested and used for perovskite-based solar cells. However, spiro-OMeTAD is moisture sensitive and causes the degradation of device performance.

It would be desirable to find and use an inorganic p-type material as the hole transport media, which would offer the double advantage of reducing the overall cost of the cell and also enhance its resistance to degradation. At present, only a few inorganic materials have been tested, such as copper iodide (CuI), copper thiocyanate (CuSCN) and nickel oxide (NiO). In order to truly replace silicon-based solar cells, an inorganic hole transport material must be found that desirably provides high carrier mobility and a defect-free interface with the absorbing layer to minimize carrier recombination.

Thus, a hybrid organic-inorganic perovskite-based solar cell with copper oxide as a hole transport material addressing the aforementioned problems is desired.

### DISCLOSURE OF INVENTION

The hybrid organic-inorganic perovskite-based solar cell with copper oxide as a hole transport material includes a transparent conducting film layer for mounting on a glass substrate or the like, and a titanium dioxide (TiO₂) layer formed on the conducting film layer. The transparent conducting film layer can be fluorine-doped tin oxide (FTO) or the like, although it should be understood that any suitable transparent conducting material may be used. A methylammonium lead halide perovskite layer, such as a lead methylammonium tri-iodide perovskite (CH₃NH₃PbI₃) layer, is formed on the titanium dioxide layer, such that the titanium dioxide layer is sandwiched between the methylammonium lead halide perovskite layer and the transparent conducting film layer. The methylammonium lead halide perovskite layer acts as a light absorber and the titanium dioxide acts as an electron transport material.

A layer of copper oxide (Cu₂O) is formed on the methylammonium lead halide perovskite layer. The copper oxide (Cu₂O) acts as hole transport material (HTM). The methylammonium lead halide perovskite layer is sandwiched between the Cu₂O layer and the titanium dioxide layer. A conductive metallic contact, such as a gold (Au) contact, is formed on the layer of hole transport material, such that the layer of hole transport material is positioned between the conductive metallic contact and the methylammonium lead halide perovskite layer.

As in a conventional solar cell, the methylammonium lead halide perovskite layer acts as the absorber and the titanium dioxide acts as an electron transport material.

These and other features of the present invention will become readily apparent upon further review of the following specification and drawings.

### BRIEF DESCRIPTION OF THE DRAWINGS

FIG. 1A is a perspective view of an embodiment of a hybrid organic-inorganic perovskite-based solar cell with copper oxide as a hole transport material according to the present invention.

FIG. 1B is an energy level diagram comparing energy band alignments of a titanium dioxide (TiO₂) layer, a lead methylammonium tri-iodide perovskite (CH₃NH₃PbI₃) layer, a copper oxide (Cu₂O) layer and a gold (Au) layer of the hybrid organic-inorganic perovskite-based solar cell with copper oxide as a hole transport material according to the present invention.

FIG. 2 is an energy level diagram comparing the energy band alignment of lead methylammonium tri-iodide perovskite (CH₃NH₃PbI₃) against those of the hole transport materials 2,2′,7,7′-tetrakis(N,N-di-p-methoxyphenylamine)-9′,9-spirobifluorene (spiro-OMeTAD), copper thiocyanate (CuSCN), nickel oxide (NiO) and copper iodide (CuI).

FIG. 3A is a graph of open circuit voltage (V_{oc}) as a function of absorber layer thicknesses in the range of 200 nanometers (nm) to 600 nm for a recombination value of 7.2×10⁻¹⁰ centimeters³/second (cm³/s) for perovskite-based solar cells using copper oxide (Cu₂O), 2,2′,7,7′-tetrakis(N,N-di-p-methoxyphenylamine)-9′,9-spirobifluorene (spiro-OMeTAD), copper thiocyanate (CuSCN), nickel oxide (NiO) and copper iodide (CuI) as hole transport materials.

FIG. 3B is a graph of short circuit current (J_{sc}) as a function of absorber layer thicknesses in the range of 200 nm to 600 nm for a recombination value of 7.2×10⁻¹⁰ cm³/s for perovskite-based solar cells using copper oxide (Cu₂O), 2,2′,7,7′-tetrakis(N,N-di-p-methoxyphenylamine)-9′,9-spirobifluorene (spiro-OMeTAD), copper thiocyanate (CuSCN), nickel oxide (NiO) and copper iodide (CuI) as hole transport materials.

FIG. 3C is a graph of fill factor (FF) as a function of absorber layer thicknesses in the range of 200 nm to 600 nm for a recombination value of 7.2×10⁻¹⁰ cm³/s for perovskite-based solar cells using copper oxide (Cu₂O), 2,2′,7,7′-tetrakis(N,N-di-p-methoxyphenylamine)-9′,9-spirobifluorene (spiro-OMeTAD), copper thiocyanate (CuSCN), nickel oxide (NiO) and copper iodide (CuI) as hole transport materials.

FIG. 3D is a graph of efficiency (η) as a function of absorber layer thicknesses in the range of 200 nm to 600 nm for a recombination value of 7.2×10⁻¹⁰ cm³/s for perovskite-based solar cells using copper oxide (Cu₂O), 2,2′,7,7′-tetrakis(N,N-di-p-methoxyphenylamine)-9′,9-spirobifluorene (spiro-OMeTAD), copper thiocyanate (CuSCN), nickel oxide (NiO) and copper iodide (CuI) as hole transport materials.

FIG. 4A is a graph of open circuit voltage (V_{oc}) as a function of absorber layer thicknesses in the range of 200 nm to 600 nm for recombination values of 7.2×10⁻⁹ cm³/s and 7.2×10⁻¹² cm³/s for the hybrid organic-inorganic perovskite-based solar cell with copper oxide as a hole transport material according to the present invention.

FIG. 4B is a graph of short circuit current (J_{sc}) as a function of absorber layer thicknesses in the range of 200 nm to 600 nm for recombination values of 7.2×10⁻⁹ cm³/s and 7.2×10⁻¹² cm³/s for the hybrid organic-inorganic perovskite-based solar cell with copper oxide as a hole transport material according to the present invention.

FIG. 4C is a graph of fill factor (FF) as a function of absorber layer thicknesses in the range of 200 nm to 600 nm for recombination values of 7.2×10⁻⁹ cm³/s and 7.2×10⁻¹² cm³/s for the hybrid organic-inorganic perovskite-based solar cell with copper oxide as a hole transport material according to the present invention.

FIG. 4D is a graph of efficiency (η) as a function of absorber layer thicknesses in the range of 200 nm to 600 nm for recombination values of 7.2×10⁻⁹ cm³/s and 7.2×10⁻¹² cm³/s for the hybrid organic-inorganic perovskite-based solar cell with copper oxide as a hole transport material according to the present invention.

FIG. 5 is a graph of saturation current (J₀) as a function of absorber layer thicknesses in the range of 200 nm to 600 nm for perovskite-based solar cells using copper oxide (Cu₂O), 2,2′,7,7′-tetrakis(N,N-di-p-methoxyphenylamine)-9′,9-spirobifluorene (spiro-OMeTAD), copper thiocyanate (CuSCN), nickel oxide (NiO) and copper iodide (CuI) as hole transport materials.

FIG. 6A is a graph of efficiency (η) as a function of defect density in the range of 1×10¹⁴ cm⁻³ to 1×10¹⁷ cm⁻³ for the hybrid organic-inorganic perovskite-based solar cell with copper oxide as a hole transport material with defects in the copper oxide but with no defects in the perovskite.

FIG. 6B is a graph of efficiency (η) as a function of defect density in the range of 1×10¹⁴ cm⁻³ to 1×10¹⁷ cm⁻³ for the hybrid organic-inorganic perovskite-based solar cell with copper oxide as a hole transport material with defects in the perovskite but with no defects in the copper oxide.

Unless otherwise indicated, similar reference characters denote corresponding features consistently throughout the attached drawings.

### BEST MODES FOR CARRYING OUT THE INVENTION

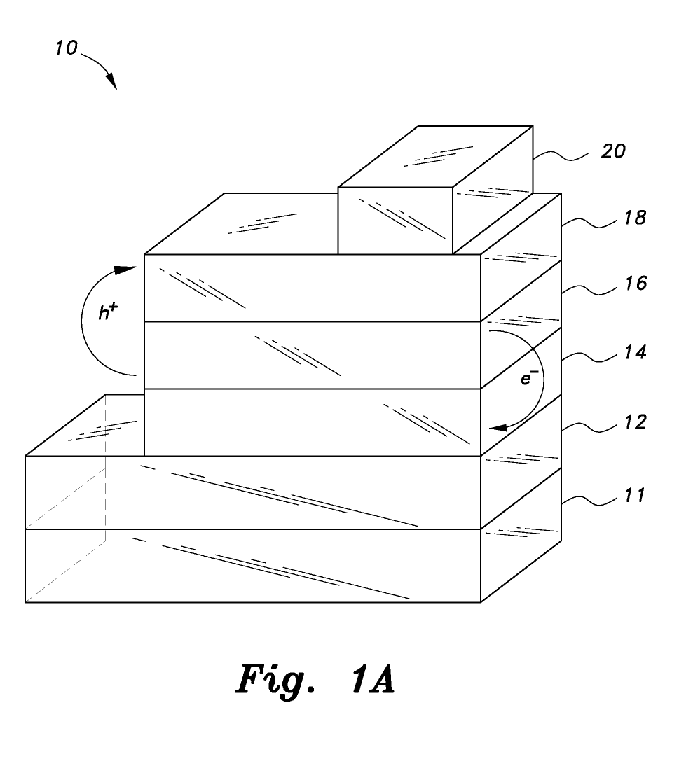

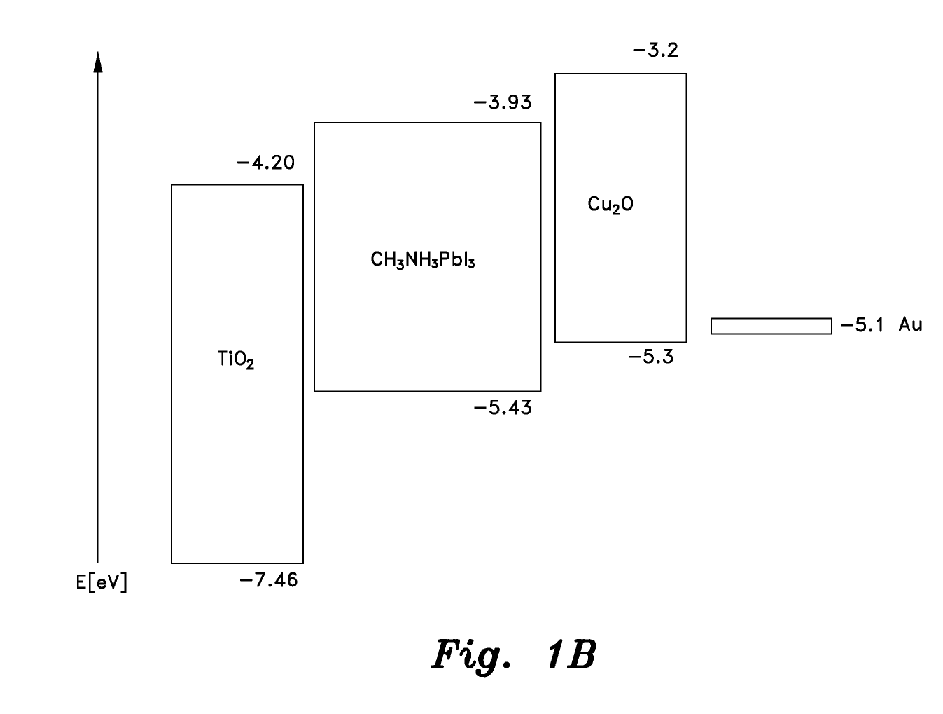

Referring now to FIG. 1A, the hybrid organic-inorganic perovskite-based solar cell with copper oxide as a hole transport material 10 includes a transparent conducting film layer 12 for mounting on a glass substrate 11, as is conventionally known in solar cells, and a titanium dioxide (TiO₂) layer 14 formed thereon. The transparent conducting film layer 12 can be a fluorine-doped tin oxide (FTO) or the like, although it should be understood that any suitable transparent conducting material may be used. A methylammonium lead halide perovskite layer, such as desirably a lead methylammonium tri-iodide perovskite (CH₃NH₃PbI₃) layer 16, is formed on the titanium dioxide layer 14, such that the titanium dioxide layer 14 is sandwiched between the lead methylammonium tri-iodide perovskite layer 16 and the transparent conducting film layer 12. The halide of the methylammonium lead halide perovskite layer can be a suitable halide, such as fluorine, chlorine or bromine, as well as iodine.

A layer of hole transport material 18 is formed on the lead methylammonium tri-iodide perovskite layer 16, the layer of the hole transport material 18 being composed of copper oxide (Cu₂O). The lead methylammonium tri-iodide perovskite layer 16 is sandwiched between the layer of hole transport material 18 and the titanium dioxide layer 14. A conductive metallic contact, such as desirably a gold (Au) contact 20, is formed on the layer of hole transport material 18, such that the layer of hole transport material 18 is positioned between the gold contact 20 and the lead methylammonium tri-iodide perovskite layer 16. As in a conventional solar cell, the lead methylammonium tri-iodide perovskite layer acts as the absorber and the titanium dioxide acts as an electron transport material.

Copper oxide, which is a p-type semiconductor, is used for the layer of hole transport material 18 due to its low electron affinity (3.2 eV) and high hole mobility. Cu₂O thin films can be prepared using a wide variety of techniques, including sputtering, copper oxidation, and atomic layer deposition (ALD). It should be noted that unintentionally doped films are naturally p-type because of the native defects identified as negatively charged copper vacancies (V′_{Cu}) rather than interstitial oxygen (O′ᵢ).

Experimental work on such materials has found hole trapping levels between 0.36 electron volts (eV) and 0.55 eV above the valence band (Eᵥ). A native defect state has also been found at 0.45 eV above the valence band assigned to copper vacancies. Nitrogen doping has been used to prepare samples containing a density of holes as high as 10¹⁸ cm⁻³. Material properties of copper oxide are shown below in Table 1, including dielectric permittivity, electron mobility, hole mobility, acceptor concentration, band gap, conduction band density of states (CB DOS), valence band density of states (VB DOS), affinity and defect level.

TABLE 1
Material Properties of Copper Oxide (Cu₂O)

| | |
|---|---|
| Dielectric Permittivity | 7.11 |
| Electron Mobility (cm²/Vs) | 200.00 |
| Hole Mobility (cm²/Vs) | 80.00 |
| Acceptor Concentration (cm⁻³) | 1.00 × 10¹⁸ |
| Band Gap (eV) | 2.17 |
| CB DOS (cm⁻³) | 2.02 × 10¹⁷ |
| VB DOS (cm⁻³) | 1.10 × 10¹⁹ |
| Affinity (eV) | 3.20 |
| Defect Level (above the edge of Eᵥ) (eV) | 0.45 |

The energy level alignment is a relatively important factor that affects the performance of the cell. Photoelectrons (e⁻) are injected from the perovskite layer 16 to the TiO₂ layer 14, and holes (h⁺) are injected from the perovskite layer 16 to the hole transport material (HTM) layer 18. The extraction of photoelectrons at the TiO₂/perovskite interface typically requires that the electron affinity (EA) of the perovskite be higher than that of the TiO₂, while the extraction of holes at the HTM/perovskite interface typically requires that the ionization energy of the HTM be lower than that of the perovskite. The energy level mismatches at both interfaces affect both the short circuit current and the open circuit voltage. The energy level diagram of an embodiment of the TiO₂/CH₃NH₃PbI₃/Cu₂O/Au solar cell 10 is shown in FIG. 1B, for example.

It should be noted that a 0.7 eV energy barrier for electrons exists at the perovskite/HTM interface and prevents the transfer of photoelectrons to the copper oxide layer. The electronic and optical properties of Cu₂O are strongly affected by point and structural defects, which are material growth dependent. The careful thickness selection of an inorganic hole transport material, like Cu₂O, can act as a capping layer, which prevents or substantially prevents contact between the perovskite and the conductive metallic contact, such as a gold contact, for example. As will be described in detail below, a numerical analysis of the cell performance was carried out first assuming defect-free Cu₂O and perovskite and, subsequently, with defects in both layers.

The numerical analysis was carried out using wx Analysis of Microelectronic and Photonic Structures (wxAMPS) software, which was developed at the University of Illinois. The software numerically solved the three main equations that govern carrier transport, namely, the Poisson and continuity equations for electrons and holes. Computations were also carried out using the solar cell capacitance simulator (SCAPS) developed at the Department of Electronics and Information Systems of the University of Gent in Belgium to consolidate the results. SCAPS captures the analytical physics of the solar cell device, including, but not limited to, transport mechanism, individual carrier current densities, electric field distributions and recombination profiles.

The parameters of materials other than Cu₂O used in the simulations are listed below in Table 2, for purposes of comparison against Cu₂O. These other materials include titanium dioxide (TiO₂), lead methylammonium tri-iodide perovskite (CH₃NH₃PbI₃), 2,2′,7,7′-tetrakis(N,N-di-p-methoxyphenylamine)-9′,9-spirobifluorene (spiro-OMeTAD), p-type copper thiocyanate (p-CuSCN), p-type nickel oxide (p-NiO) and p-type copper iodide (p-CuI).

TABLE 2
Material Properties of TiO₂, CH3NH3PbI3, and Four Types of HTMs

| | TiO₂ | CH₃NH₃PbI₃ | Spiro-OMeTAD | p-CuSCN | p-NiO | p-CuI |
|---|---|---|---|---|---|---|
| Dielectric Permittivity | 10 | 10 | 3 | 10 | 10.7 | 6.5 |
| Electron Mobility (cm²/Vs) | 100 | 100 | 1 × 10⁻⁴ | 100 | 12 | 100 |
| Hole Mobility (cm²/Vs) | 25 | 10 | 2 × 10⁻⁴ | 25 | 2.8 | 43.9 |
| Acceptor Concentration | 0 | 1 × 10⁹ | 1 × 10¹⁸ | 1 × 10¹⁸ | 1 × 10¹⁸ | 1 × 10¹⁸ |
| (cm⁻³) | | | | | | |
| Donor Concentration | 1 × 10¹⁷ | 1 × 10⁹ | 0 | 0 | 0 | 0 |
| (cm⁻³) | | | | | | |
| Band Gap (eV) | 3.26 | 1.5 | 3.06 | 3.6 | 3.8 | 3.1 |
| CB DOS (cm⁻³) | 2 × 10¹⁷ | 2.75 × 10¹⁸ | 2.8 × 10¹⁹ | 2.2 × 10¹⁹ | 2.8 × 10¹⁹ | |
| VB DOS (cm⁻³) | 6 × 10¹⁷ | 3.9 × 10¹⁸ | 1 × 10¹⁹ | 1.8 × 10¹⁸ | 1 × 10¹⁹ | 1 × 10¹⁹ |
| Affinity (eV) | 4.2 | 3.9 | 2.05 | 1.7 | 1.46 | 2.1 |
| Band-to-Band | 7.2 × 10⁻⁹ | | 7.2 × 10⁻¹⁰ | | 7.2 × 10⁻¹¹ | |
| Recombination Rate | | | | | | |
| (cm³/s) | | | | | | |

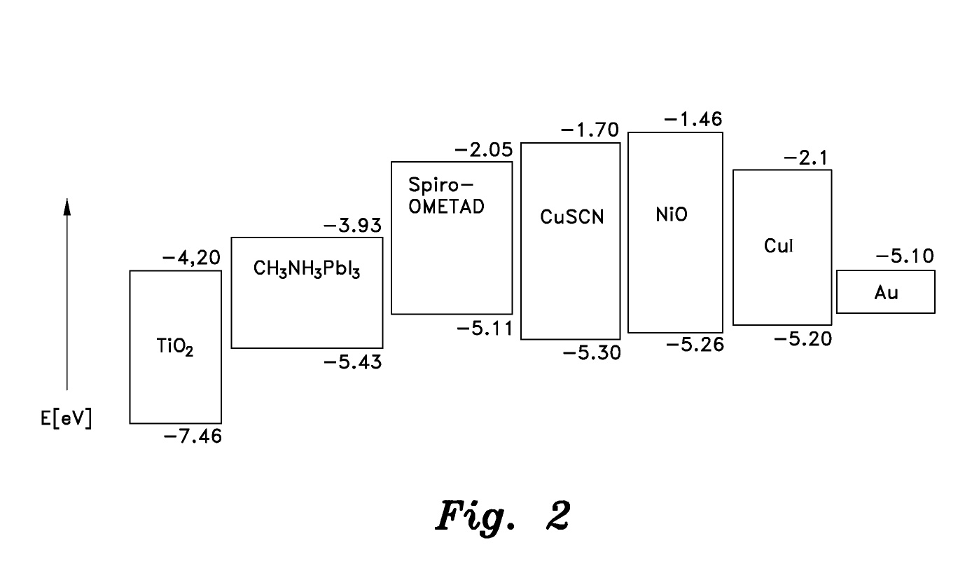

The effective density of states in the conduction and valence bands of perovskite, as shown above in Table 2, have been calculated using the effective masses obtained from electronic structure calculations on the pseudo-cubic phase, where the electron effective mass, mₑ*/mₒ=0.23 and the hole effective mass, mₕ*/mₒ=0.29, where m₀ is the free electron rest mass and typically m₀=9.11×10⁻³¹ kg. In the absence of a measured value of the band to band recombination rate, a value of 7.20×10⁻¹⁰ cm³/s has been used, which is a value that has been reported for GaAs in the literature, which has a direct gap of 1.43 eV. FIG. 2 shows the energy band alignment of the various materials used in the simulations.

Initially, the HTM layer, absorber, and TiO₂ layer thicknesses were considered to be defect free. The initial values of thicknesses of the absorber and the TiO₂ layer, as the electron transport material (ETM) layer, were set to an initial value, the initial values being equal to 300 nm and 100 nm, respectively. These values are found to be optimum values for typical perovskite-based cells using spiro-OMeTAD as the HTM. The iteration process was launched to optimize the HTM thickness, such as for an efficiency (ηₘₐₓ) of a solar cell. Then, using the obtained optimum value for the HTM, iterations were carried out to compute the new optimum TiO₂ layer thickness as the ETM layer thickness, such as for ηₘₐₓ. The process was repeated numerous times to determine the optimum set of thicknesses values (TiO₂ (ETM), HTM) for the five cell structures under consideration.

Finally, the optimized values for the HTM and the TiO₂ layer were used to compute the optimum thickness of the absorbing layer, such as for ηₘₐₓ. The optimized values for the three layers obtained through the iteration process are shown below in Table 3. The optimum values are about 150 nm for the TiO₂ layer (i.e., the electron transport material (ETM) layer) and the HTM layer, and in the range of 350 nm-450 nm for the perovskite absorber layer.

TABLE 3
Optimized Thicknesses of ETM, Absorber and HTM Layers

| HTM | ETM | Absorber | HTM |
|---|---|---|---|
| Type | Thickness (nm) | Thickness (nm) | Thickness (nm) |
| Cu₂O | 140 | 350 | 150 |
| Spiro- | 140 | 450 | 150 |
| OMeTAD | | | |
| CuSCN | 145 | 450 | 200 |
| NiO | 135 | 450 | 200 |
| CuI | 145 | 400 | 200 |

The key characteristics of the solar cells were computed using both software, as described, and considering the optimized values of the thicknesses of different layers reported in Table 3 above. These characteristics include the fill factor (FF), the open circuit voltage (V_{oc}), the short circuit current density (J_{sc}) and the power conversion Efficiency (PCE) corresponding to different types of HTMs. The obtained values are compiled below in Tables 4A and 4B. The results clearly show that the device using Cu₂O as the hole transport material has the highest performance

TABLE 4
Optimized Performances for HTMs

| HTM | V_{oc} (volts (V)) | | | J_{sc} (milliamps (mA)/cm²)) | | |
|---|---|---|---|---|---|---|
| Type | SCAPS | wxAMPS | Exp. | SCAPS | wxAMPS | Exp. |
| Cu₂O | 1.276 | 1.249 | — | 22.75 | 24.76 | — |
| Spiro- | 1.214 | 1.226 | 0.993 | 21.84 | 24.17 | 20.0 |
| OMeTAD | | | | | | |
| CuSCN | 1.295 | 1.281 | 1.016 | 20.63 | 23.05 | 19.7 |
| NiO | 1.125 | 1.178 | 0.936 | 20.24 | 21.87 | 14.9 |
| CuI | 1.092 | 1.129 | 0.55 | 21.32 | 23.09 | 17.8 |

TABLE 5
Optimized Performances for HTMs

| HTM | FF (%) | | | PCE (%) | | |
|---|---|---|---|---|---|---|
| Type | SCAPS | wxAMPS | Exp. | SCAPS | wxAMPS | Exp. |
| Cu₂O | 83.97 | 83.54 | — | 24.40 | 25.86 | — |
| Spiro- | 81.48 | 80.02 | 73 | 21.97 | 22.52 | 15.0 |
| OMeTAD | | | | | | |
| CuSCN | 80.71 | 79.11 | 62 | 22.03 | 23.38 | 12.4 |
| NiO | 82.38 | 80.78 | 75 | 19.19 | 20.81 | 7.26 |
| CuI | 81.69 | 80.04 | 60 | 19.43 | 20.87 | 6.0 |

From Tables 4A and 4B, it can be noted that the PCE values obtained by wxAMPS are slightly higher than those obtained by SCAPS, but the two software packages provide consistent ranking of the calculated performances for different HTMs: Cu₂O (highest performance), CuSCN, spiro-OMeTAD, CuI, then NiO (lowest performance). It should be noted that the experimental values (Exp.) are significantly lower than the simulated ones, especially in the cases of NiO and CuI. This is expected because point defects in the bulk of the absorbing layer, as well as at the TiO₂/perovskite and perovskite/HTM interfaces, can act as recombination centers that lower both the collected current and the open circuit voltage.

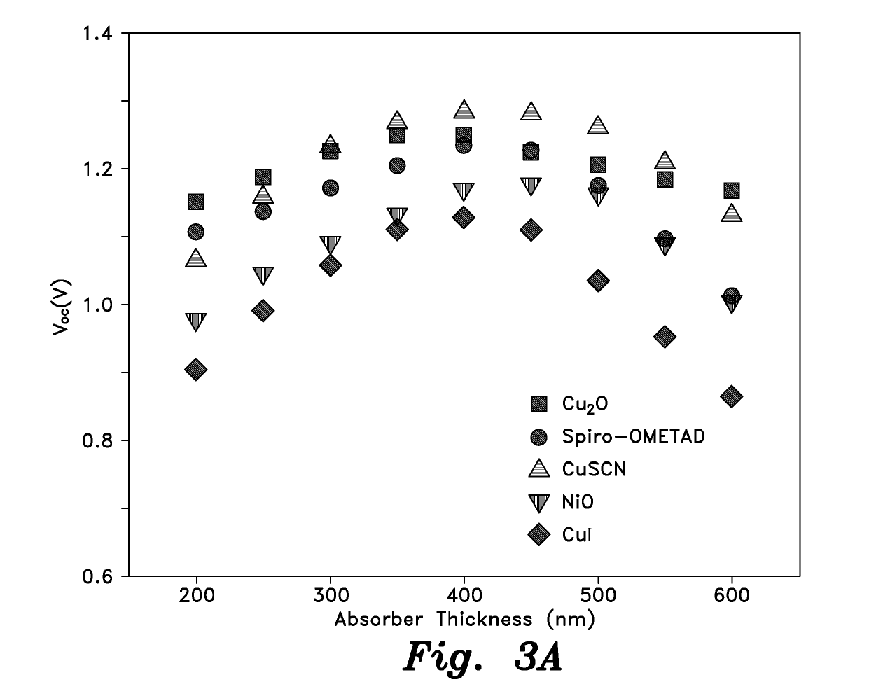

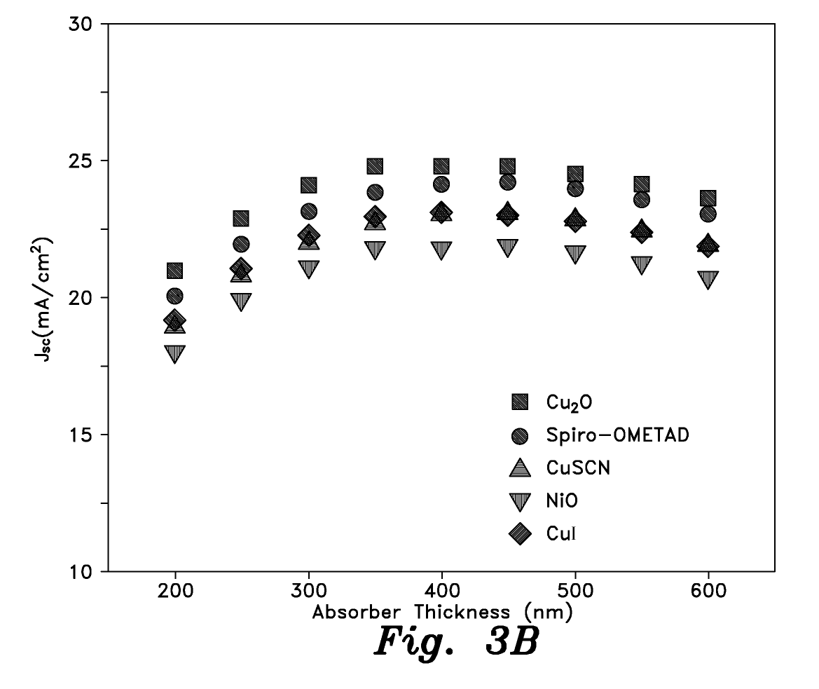

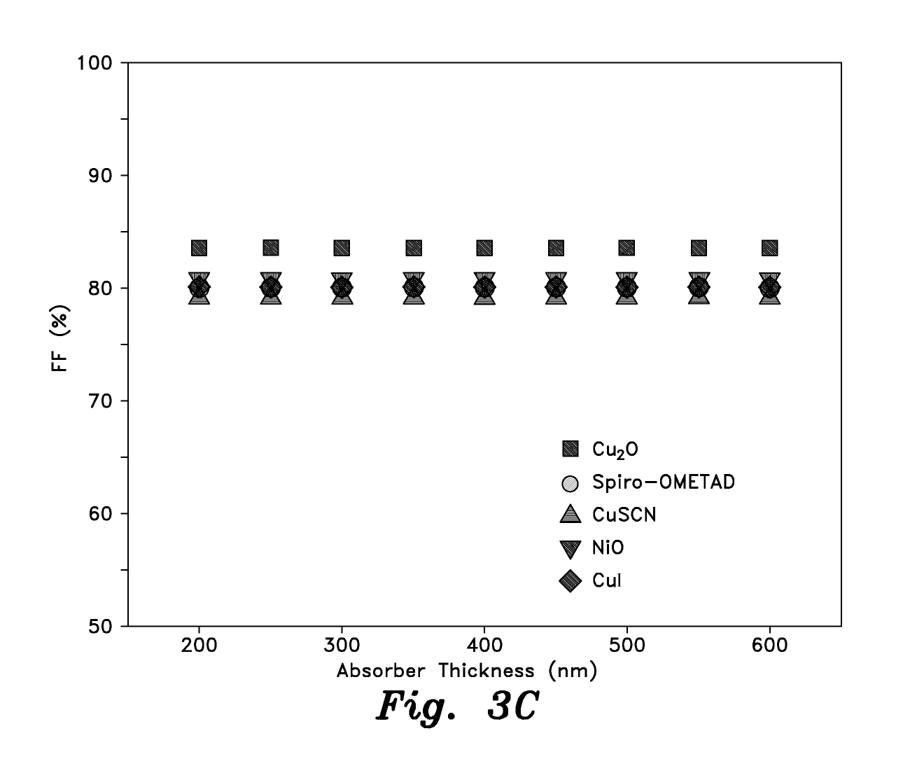

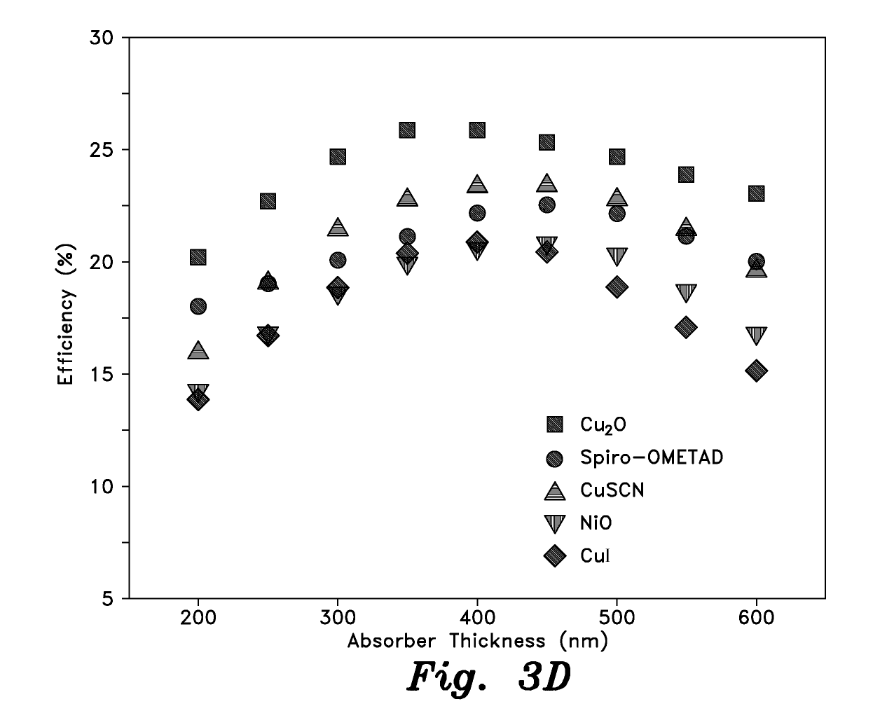

Lead halide perovskites are characterized by a high light absorbance and a relatively high carrier diffusion length reaching one micron. A layer of a few hundred nanometer thickness is enough to absorb most of the incident sun light, for example. Therefore, most of the photocarriers can be collected, as they are generated at distances less than the diffusion length away from the perovskite/TiO₂ and perovskite/HTM interfaces. Additionally, the variation of the parameters that determine the solar cell efficiency as a function of the absorber layer thickness in the range of 200 nm-600 nm has been calculated using wxAMPS. The results are shown in FIGS. 3A-3D.

FIG. 3A is a graph of open circuit voltage (V_{oc}) as a function of absorber layer thicknesses in the range of 200 nm to 600 nm for a recombination value of 7.2×10⁻¹⁰ cm³/s for perovskite-based solar cells using copper oxide (Cu₂O), 2,2′,7,7′-tetrakis(N,N-di-p-methoxyphenylamine)-9′,9-spirobifluorene (spiro-OMeTAD), copper thiocyanate (CuSCN), nickel oxide (NiO) and copper iodide (CuI) as hole transport materials.

FIG. 3B is a graph of short circuit current (J_{sc}) as a function of absorber layer thicknesses in the range of 200 nm to 600 nm for a recombination value of 7.2×10⁻¹⁰ cm³/s for perovskite-based solar cells using copper oxide (Cu₂O), 2,2′,7,7′-tetrakis(N,N-di-p-methoxyphenylamine)-9′,9-spirobifluorene (spiro-OMeTAD), copper thiocyanate (CuSCN), nickel oxide (NiO) and copper iodide (CuI) as hole transport materials.

FIG. 3C is a graph of fill factor (FF) as a function of absorber layer thicknesses in the range of 200 nm to 600 nm for a recombination value of 7.2×10⁻¹⁰ cm³/s for perovskite-based solar cells using copper oxide (Cu₂O), 2,2′,7,7′-tetrakis(N,N-di-p-methoxyphenylamine)-9′,9-spirobifluorene (spiro-OMeTAD), copper thiocyanate (CuSCN), nickel oxide (NiO) and copper iodide (CuI) as hole transport materials.

FIG. 3D is a graph of efficiency (η) as a function of absorber layer thicknesses in the range of 200 nm to 600 nm for a recombination value of 7.2×10⁻¹⁰ cm³/s for perovskite-based solar cells using copper oxide (Cu₂O), 2,2′,7,7′-tetrakis(N,N-di-p-methoxyphenylamine)-9′,9-spirobifluorene (spiro-OMeTAD), copper thiocyanate (CuSCN), nickel oxide (NiO) and copper iodide (CuI) as hole transport materials.

In order to confirm that the decrease of V_{oc} and PCE beyond 400 nm is related to current loss due to carrier recombination, simulations were also performed using two different values of the recombination constant, B, namely values of 7.2×10⁻⁹ cm³/s and 7.2×10⁻¹² cm³/s.

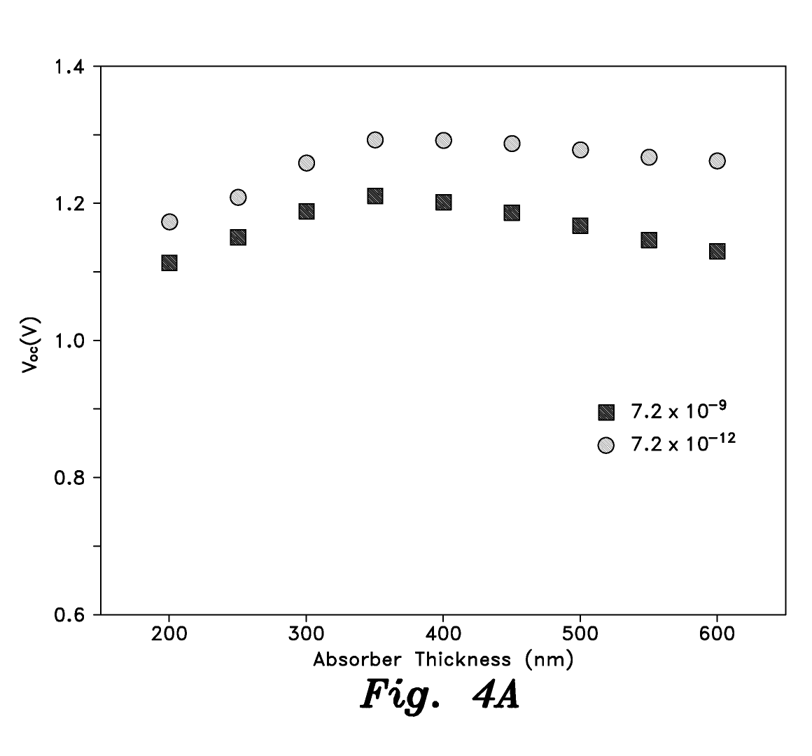

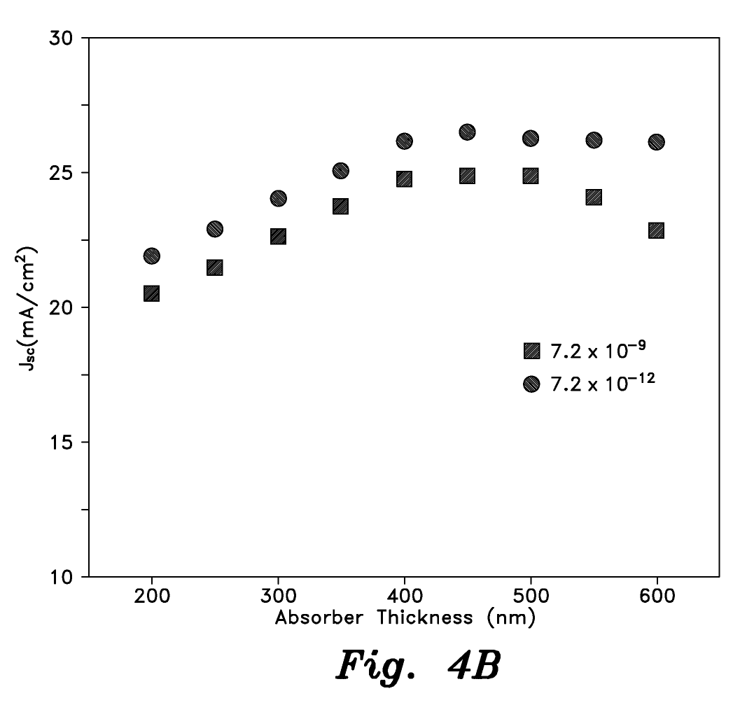

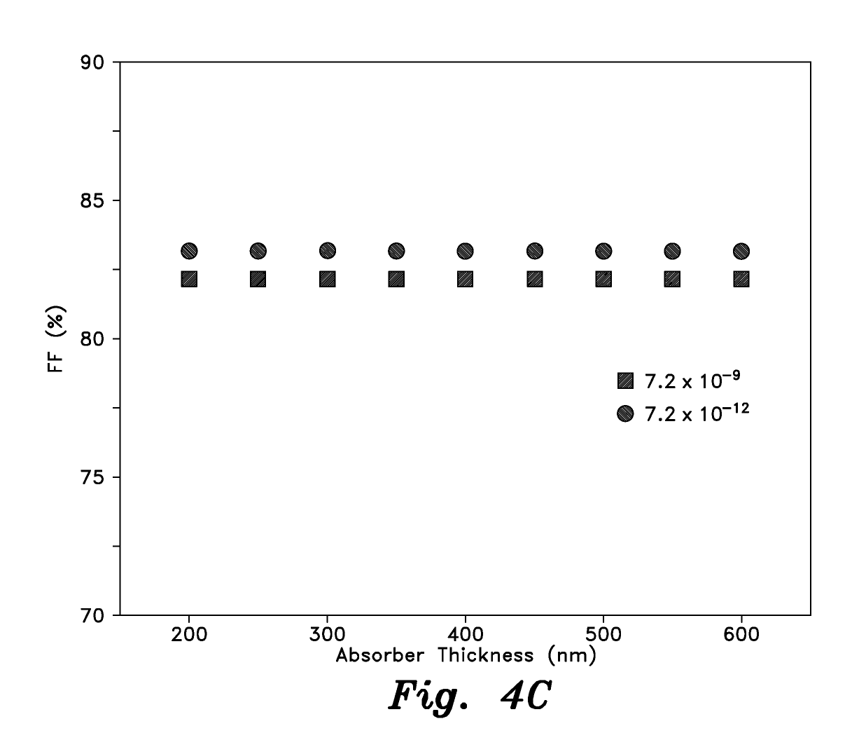

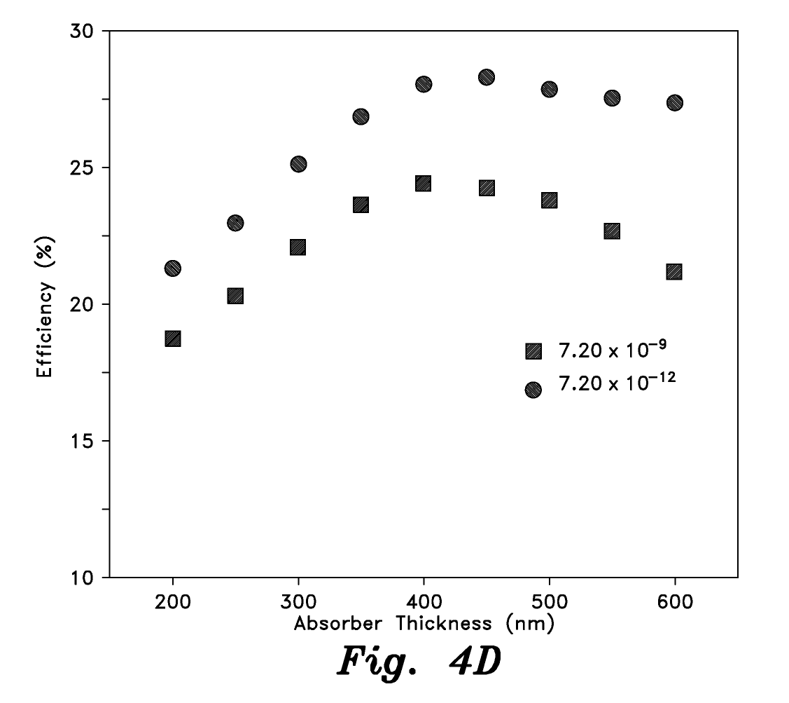

FIG. 4A is a graph of open circuit voltage (V_{oc}) as a function of absorber layer thicknesses in the range of 200 nm to 600 nm for recombination values of 7.2×10⁻⁹ cm³/s and 7.2×10⁻¹² cm³/s for the hybrid organic-inorganic perovskite-based solar cell with copper oxide as a hole transport material.

FIG. 4B is a graph of short circuit current (J_{sc}) as a function of absorber layer thicknesses in the range of 200 nm to 600 nm for recombination values of 7.2×10⁻⁹ cm³/s and 7.2×10⁻¹² cm³/s for the hybrid organic-inorganic perovskite-based solar cell with copper oxide as a hole transport material.

FIG. 4C is a graph of fill factor (FF) as a function of absorber layer thicknesses in the range of 200 nm to 600 nm for recombination values of 7.2×10⁻⁹ cm³/s and 7.2×10⁻¹² cm³/s for the hybrid organic-inorganic perovskite-based solar cell with copper oxide as a hole transport material.

FIG. 4D is a graph of efficiency (η) as a function of absorber layer thicknesses in the range of 200 nm to 600 nm for recombination values of 7.2×10⁻⁹ cm³/s and 7.2×10⁻¹² cm³/s for the hybrid organic-inorganic perovskite-based solar cell with copper oxide as a hole transport material.

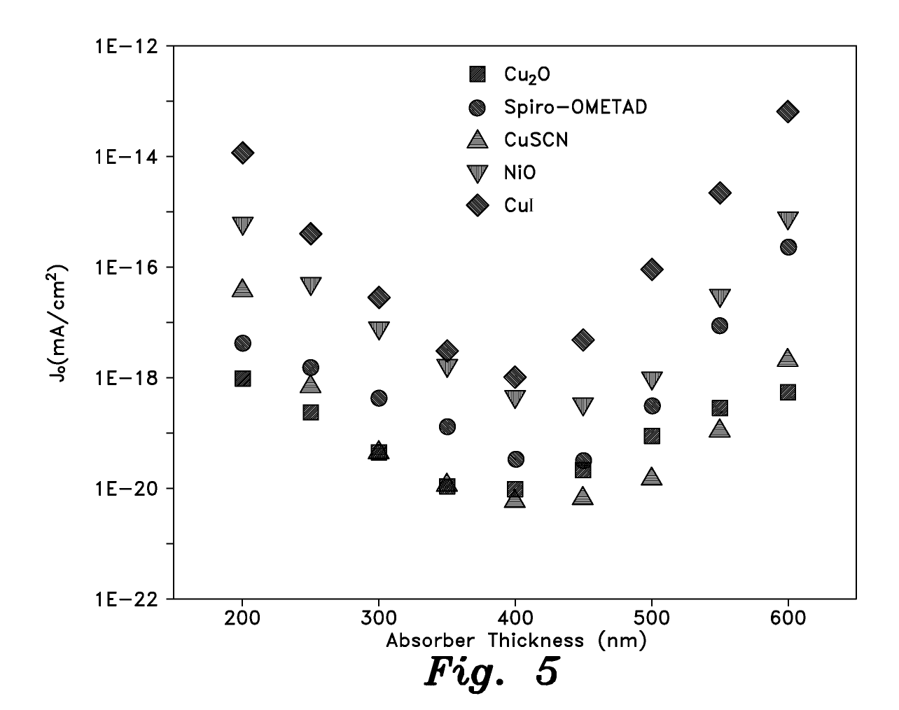

FIG. 5 shows the value of the saturation current J₀ versus the thickness of the absorbing layers. It can be clearly seen that the variation of J₀ as a function of the absorber thickness is much more significant than that of J_{sc}. Therefore, the dependence of V_{oc} versus the perovskite thickness is mainly determined by the dependence of the saturation current of the device.

Further, FIG. 3C shows that FF remains quasi-constant as the thickness of the HTM layer varies. The fill factor of Cu₂O is relatively closer to that of other HTM devices, which indicates that the highest efficiency obtained for the Cu₂O-based device is mainly due to higher values of the short circuit current and open circuit voltage.

The above numerical simulations were carried out assuming defect-free materials. In reality, various point defects are typically always present in the bulk and interfaces of perovskite-based cells, which are multi-layered devices. Thus, simulations have also been performed for the presence of select point defects. A single recombination center located at 0.45+Eᵥ was used for the p-type Cu₂O HTM layer, along with a perovskite defect level located at 0.05+Eᵥ. First, the effect of an increasing defect concentration in the Cu₂O layer was calculated, assuming a defect-free perovskite. Then, a similar calculation was performed for an increasing density of defects in perovskite, while the Cu₂O layer was assumed defect-free.

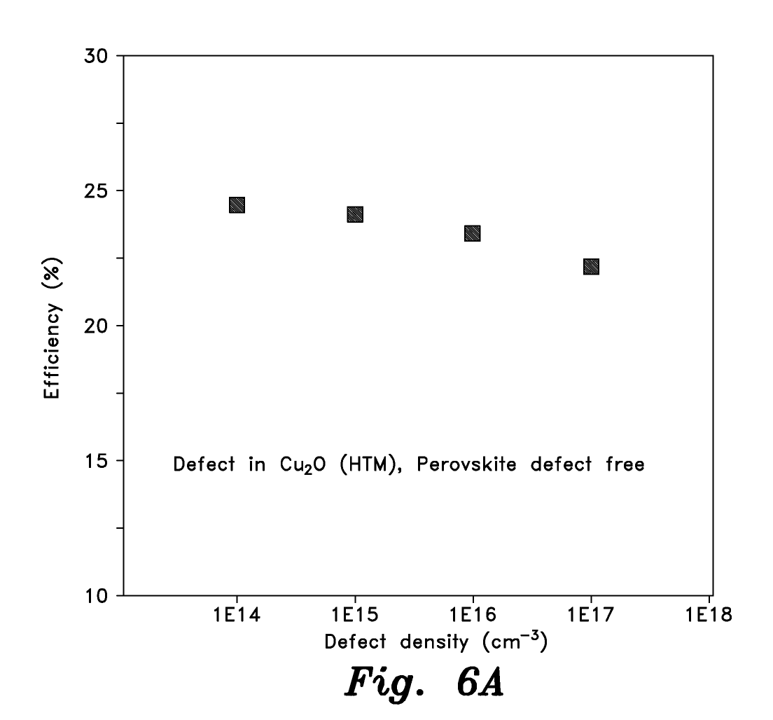

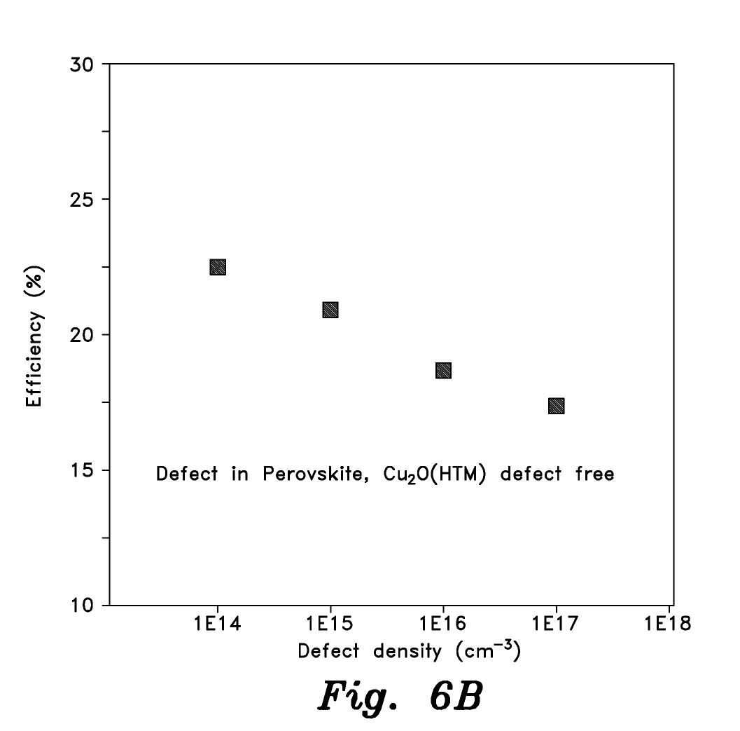

The results of the simulations are shown in FIGS. 6A and 6B, respectively. It can be seen that all cell parameters are sensitive to high defect density in the absorbing layer. The efficiency reduction is mainly due to a reduction of short circuit current, open circuit voltage and fill factor. Further, it can be seen that the considered defect state in p-Cu₂O has a smaller effect on the device performance as compared to the defect considered in the absorbing layer. This is expected, since most of the light is absorbed in the perovskite layer. Thus, a more significant effect is expected on the carrier lifetime and the recombination rate.

It is to be understood that the present invention is not limited to the embodiments described above, but encompasses any and all embodiments within the scope of the following claims.

We claim:

1. A hybrid organic-inorganic perovskite-based solar cell with copper oxide as a hole transport material, comprising:
a glass substrate;
a transparent conducting film layer formed on the glass substrate;
a layer of electron transport material formed on the transparent conducting film layer such that the transparent conducting film layer is sandwiched between the glass substrate and the layer of electron transport material;
a light absorber layer formed on the electron transport material layer, the layer of electron transport material being sandwiched between the light absorber layer and the transparent conducting film layer;
a layer of hole transport material formed on the light absorber layer, the hole transport material including copper oxide, the light absorber layer being sandwiched between the layer of hole transport material and the layer of electron transport material; and
a conductive metallic contact formed on the layer of hole transport material, the layer of hole transport material being positioned between the conductive metallic contact and the light absorber layer.

2. The hybrid organic-inorganic perovskite-based solar cell as recited in claim 1, wherein the conductive metallic contact comprises gold.

3. The hybrid organic-inorganic perovskite-based solar cell as recited in claim 1, wherein said layer of electron transport material comprises titanium dioxide.

4. The hybrid organic-inorganic perovskite-based solar cell as recited in claim 1, wherein said transparent conducting film layer comprises fluorine-doped tin oxide.

5. The hybrid organic-inorganic perovskite-based solar cell as recited in claim 1, wherein said light absorber layer comprises a methylammonium lead halide perovskite.

6. The hybrid organic-inorganic perovskite-based solar cell as recited in claim 1, wherein said light absorber layer comprises lead methylammonium tri-iodide perovskite.

7. The hybrid organic-inorganic perovskite-based solar cell as recited in claim 1, wherein the layer of electron transport material has a thickness of about 150 nm.

8. The hybrid organic-inorganic perovskite-based solar cell as recited in claim 7, wherein the layer of hole transport material has a thickness of about 150 nm.

9. The hybrid organic-inorganic perovskite-based solar cell as recited in claim 8, wherein the light absorber layer has a thickness between 350 nm and 450 nm.

10. A hybrid organic-inorganic perovskite-based solar cell, comprising:
a substrate;
a multi-layer semiconductor having:
a transparent conducting film layer disposed on the substrate;
a layer of titanium dioxide disposed on the transparent conducting film layer dioxide for electron transport;
a layer of a methylammonium lead halide perovskite-based material disposed on the layer of titanium dioxide for absorbing light;
a layer of copper oxide (Cu₂O) disposed on the layer of a methylammonium lead halide perovskite-based material for hole transport; and
a conductive metallic contact disposed on the layer of copper oxide.

11. The hybrid organic-inorganic perovskite-based solar cell according to claim 10, wherein said layer of a methylammonium lead halide perovskite-based material comprises a layer of lead methylammonium tri-iodide perovskite.

12. The hybrid organic-inorganic perovskite-based solar cell according to claim 10, wherein said transparent conducting film layer comprises a layer of fluorine-doped tin oxide.

13. The hybrid organic-inorganic perovskite-based solar cell according to claim 10, wherein said layer of copper oxide is a thin film layer having a thickness of about 150 nm.

14. The hybrid organic-inorganic perovskite-based solar cell according to claim 13, wherein said layer of a methylammonium lead halide perovskite-based material has a thickness between 350 nm and 450 nm.

15. The hybrid organic-inorganic perovskite-based solar cell according to claim 14, wherein said layer of titanium dioxide has a thickness of about 150 nm.
